(19) United States
(12) Patent Application Publication
(10) Pub. No.: US 2017/0146453 A1
(43) Pub. Date: May 25, 2017

(54) SINGLE CHANNEL TERAHERTZ ENDOSCOPY

(71) Applicant: University of Massachusetts, Boston, MA (US)

(72) Inventors: Robert H. Giles, Upton, MA (US); Pallavi Doradla, Lowell, MA (US); Cecil S. Joseph, Lowell, MA (US)

(73) Assignee: University of Massachusetts, Boston, MA (US)

(21) Appl. No.: 15/423,711

(22) Filed: Feb. 3, 2017

Related U.S. Application Data
(63) Continuation of application No. PCT/US2015/043933, filed on Aug. 6, 2015.
(60) Provisional application No. 62/033,980, filed on Aug. 6, 2014.

(51) Int. Cl. G01N 21/3581; A61B 5/05; A61B 5/00; G02B 23/24; A61B 1/06

(52) U.S. Cl. CPC G01N 21/3581; G02B 23/2407; A61B 1/0669; A61B 5/0084; A61B 5/0507

(57) ABSTRACT

A terahertz endoscopic system including a flexible waveguide and system optics. The waveguide is configured to transmit terahertz radiation from a first end of the waveguide to a second end of the waveguide proximate to a sample, and transmit reflected terahertz radiation from the second end of the waveguide to the first end of the waveguide, wherein the reflected terahertz radiation is a portion of the terahertz radiation reflected by the sample towards the second end of the waveguide. The system optics are configured to direct the terahertz radiation from a radiation source into the first end of the waveguide, isolate the reflected terahertz radiation from other radiation, and direct the reflected terahertz radiation from the first end of the waveguide to a terahertz radiation detector.

### CROSS-REFERENCE TO RELATED APPLICATIONS

This application is a continuation of International Application Serial No. PCT/US2015/043933, filed Aug. 6, 2015, which claims benefit under 35 U.S.C. §119(e) of U.S. Application Ser. No. 62/033,980, filed Aug. 6, 2014, each of which is hereby incorporated by reference in its entirety.

### BACKGROUND

Embodiments of this application relate generally to terahertz endoscopy and more specifically to single channel terahertz endoscopy.

Rapid development in terahertz source and receiver technologies has enabled applications in the fields of imaging and spectroscopy. The terahertz (THz) frequency regime, located midway between the microwave and infrared regions, has become increasingly important for biological applications due to its nonionizing nature and sensitivity to water content. Notably, THz imaging techniques have been used to detect intrinsic contrast between cancerous and normal tissues based on water content combined with structural changes (See, for example, P. Doradla, et al., “Detection of colon cancer by continuous-wave terahertz polarization imaging technique,” J. Biomed. Opt. Lett. 18(9), 0905041-3 (2013) and C. S. Joseph, et al., “Imaging of ex vivo nonmelanoma skin cancers in the optical and terahertz spectral regions,” J. Biophotonics, DOI 10.1002 (2012), both publications incorporated by reference in their entirety).

Endoscopy is a minimally invasive diagnostic medical procedure to examine the interior surfaces of an organ or tissue without the need for surgery. Besides conventional endoscopy, computed tomography (CT), magnetic resonance imaging (MRI) and positron emission tomography (PET) are conventional diagnostic imaging modalities for the detection of local and distant relapse of cancers. CT is a noninvasive technique that provides quick tomographic images of the tissue, but it uses a series of cross sectional x-rays that are ionizing and cannot detect tumors smaller than approximately 5 mm in diameter. MRI is very sensitive in detecting lesions larger than 10 mm, but uses liquid enema for contrast which is an expensive procedure. Though PET provides high sensitivity and specificity, it presents poor resolution unless the tumor is metabolically active. As terahertz rays are nonionizing and offer intrinsic contrast between normal and abnormal tissue, a THz endoscope can be used as a potential tool in the examination and detection of cancerous or precancerous regions of biological tissue.

Previously proposed THz endoscopes fall into two categories. The first category places the THz radiation source such as photo-conductive antenna at the end of the endoscope and is inserted into the patient (See, for example, Y. B. Ji, et al., “A miniaturized fiber-coupled terahertz endoscope system,” Opt. Express 17 (19), 17082-17087 (2009)). Consequently, electrical connections to drive the THz radiation source must be inserted into the patient. The second category requires multiple waveguide channels, including a first waveguide for guiding radiation to the sample and a second waveguide for guiding reflected light to a detector.

### SUMMARY

The inventors have recognized and appreciated that reducing the size of a terahertz endoscopic system would improve comfort of the patient while making the device easier to use than existing endoscopes. The inventors have recognized and appreciated at least two ways to reduce the size of a terahertz endoscope. First, the THz radiation source and the THz radiation detector should be located remotely such that neither is required to be inserted into the patient. Second, the THz radiation should be directed both to and from the patient using the same waveguide such that there is only a single channel in the endoscopic system.

Accordingly, some embodiments are directed to a terahertz endoscopic system, comprising a flexible waveguide and system optics. The waveguide is configured to: transmit terahertz radiation from a first end of the waveguide to a second end of the waveguide proximate to a sample; and transmit reflected terahertz radiation from the second end of the waveguide to the first end of the waveguide, wherein the reflected terahertz radiation is a portion of the terahertz radiation reflected by the sample towards the second end of the waveguide. The system optics are configured to direct the terahertz radiation from a radiation source into the first end of the waveguide; isolate the reflected terahertz radiation from other radiation; and direct the reflected terahertz radiation from the first end of the waveguide to a terahertz radiation detector.

In some embodiments the system optics include a first polarizer, positioned between the radiation source and the waveguide, configured to transmit radiation a first polarization; and a second polarizer, positioned between the waveguide and the terahertz radiation detector, configured to transmit radiation of a second polarization that is orthogonal to the first polarization. A beam splitter may be included to direct the reflected terahertz radiation from the first end of the waveguide to the second polarizer.

In some embodiments, the radiation source is located remotely from the second end of the waveguide and/or the terahertz radiation detector is located remotely from the second end of the waveguide.

In some embodiments, the waveguide is a flexible fiber comprising a hollow core. The waveguide may include a metallic layer positioned a first radial distance from a center of the hollow core such that the metallic layer radially surrounds the hollow core. The waveguide may also include a first nonmetallic layer positioned a second radial distance from a center of the hollow core such that the first nonmetallic layer radially surrounds the hollow core, wherein the second radial distance is greater than the first radial distance. The first nonmetallic layer may include a polymer, such as polycarbonate or polyethylene/polytetrafluoroethylene or Teflon. The waveguide may also include a second nonmetallic layer positioned a third radial distance from a center of the hollow core such that the second nonmetallic layer radially surrounds the hollow core, wherein the second radial distance is less than the first radial distance. The second nonmetallic layer may include a polymer with a low extinction or absorption coefficient, such as polystyrene.

In some embodiments, the waveguide includes a lens at the second end to achieve high-resolution imaging. The lens may be, for example, a hyper hemi-spherical lens, a hemispherical lens or a ball lens. The lens may include a hydrophobic surface.

In some embodiments, the terahertz radiation produced by the radiation source is frequency chirped.

Some embodiments are directed to a method of performing terahertz endoscopy. The method may include acts of directing terahertz radiation emitted from a radiation source into the first end of a waveguide using system optics; transmitting terahertz radiation from the first end of the waveguide to a second end of the waveguide proximate to a sample; transmitting reflected terahertz radiation from the second end of the waveguide to the first end of the waveguide, wherein the reflected terahertz radiation is a portion of the terahertz radiation reflected by the sample towards the second end of the waveguide; isolating the reflected terahertz radiation from other radiation; and directing the reflected terahertz radiation from the first end of the waveguide to a terahertz radiation detector.

In some embodiments, the act of isolating the reflected terahertz radiation from the other radiation comprises: passing the terahertz radiation through a first polarizer before directing the terahertz radiation into the first end of the waveguide, where the first polarizer transmits radiation of a first polarization; and passing the reflected terahertz radiation through a second polarizer before directing the reflected terahertz radiation to the terahertz radiation detector, wherein the second polarizer is configured to transmit radiation of a second polarization that is different from first polarization.

In some embodiments, the terahertz radiation produced by the radiation source is frequency chirped; and the act of isolating the reflected terahertz radiation from the other radiation comprises range gating the signal generated by the terahertz radiation detector

The foregoing is a non-limiting summary of the invention, which is defined by the attached claims.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic diagram of a THz endoscopic system 100 according to some embodiments;

FIG. 2A is a more detailed schematic of a THz endoscopic system 100 according to some embodiments;

FIG. 2B illustrates the THz radiation beam waist measurements in both transmission and reflection modalities;

FIG. 2C illustrates the measurement of a standard resolution target using the system 100 of FIG. 2A;

FIG. 3 is a cross sectional view of a flexible waveguide according to some embodiments;

FIG. 4 is a flow chart of a method of performing terahertz spectroscopy according to some embodiments;

FIG. 5 shows digital photographs and THz images of various objects imaged using the system 100 of FIG. 2A;

FIG. 6 shows digital photographs and THz images of various objects imaged using the system 100 of FIG. 2A; and

FIG. 7 shows digital photographs of both normal and cancerous colon tissue and cross polarized terahertz images of both normal and cancerous colon tissue.

### DETAILED DESCRIPTION

The inventors have recognized and appreciated that a single channel endoscope may be used to transmit radiation to and from the sample using a flexible, hollow, metal-coated waveguide with low propagation and bending losses with Gaussian mode preservation (See, for example, P. Doradla and R. H. Giles, “Dual-frequency characterization of bending loss in hollow flexible terahertz waveguides,” Proc. SPIE 8985 (2014) and P. Doradla, et al., “Propagation loss optimization in metal/dielectric coated hollow flexible THz waveguides,” Proc. SPIE 8261 (2012), both publications incorporated by reference in their entirety). In some embodiments, the waveguide is a metal-coated THz waveguide which, when compared to conventional uncoated polymer tubes, provides higher inner surface reflectivity at THz wavelengths. The high reflectivity results in increased confinement within the waveguide, which reduces bending loss. Based on the ratio of fiber inner diameter to the incident wavelength, the flexible waveguide preserves the polarization of the light coupled into the fiber, even at higher bending angles. Applications of a THz endoscopic device according to some embodiments include the ability to apply continuous-wave terahertz polarization-sensitive imaging techniques to areas of the body that require endoscopic access. The single channel device, according to some embodiments, can be easily integrated with existing optical endoscopic systems to provide access to intrinsic contrast between normal and diseased tissue in a way that was not previously possible.

The inventors have recognized and appreciated that intrinsic contrast between abnormal and normal tissue based on terahertz reflectivity values may be imaged using the single channel endoscopic system of some embodiments. The simple and robust THz endoscope, according to some embodiments, uses polarization sensitive reflection-based detection. Because embodiments herein utilize light reflected from a sample, rather than light transmitted through a sample, in vivo imaging may be achieved and problems associated with high absorption rate of THz radiation in tissue is avoided. Moreover, using THz imaging allows measurements to be performed without the need for contrast agents. Additionally, utilizing the cross-polarized component of the THz radiation reflected from the sample not only results in reduction of the Fresnel reflection from the sample interface but also achieves a constant terahertz reflectance ratio of normal versus cancerous tissue that is independent of the patient. For example, in a set of experiments performed in P. Doradla, et al., “Detection of colon cancer by continuous-wave terahertz polarization imaging technique,” J. Biomed. Opt. Lett. 18(9), 0905041-3 (2013), the reflectance ration of four different cancerous samples was determined for co-polarized light and cross-polarized light at 584 GHz, resulting in the following finding:

| Sample # | Co-polarization | Cross-polarization |
|---|---|---|
| Set 1 | 0.153% | 7.74% |
| Set 2 | 0.303% | 7.74% |
| Set 3 | 0.156% | 7.75% |
| Set 4 | 0.244% | 7.30% |

Notably, while the co-polarized reflectance ratio varies between samples, the cross-polarized reflectance ratio is the same for every sample. This constant reflectance ratio is preserved when tissue is observed using endoscopic techniques. Accordingly, the reflectance ratio of normal versus cancerous tissue is a useful value to measure for diagnostic purposes.

Using experimental terahertz images, comparing the cancerous areas to adjacent normal areas of the same subject yields good specificity. To quantify the reflectivity values, the relative difference in the reflected intensity between cancerous and normal areas of the same subject was calculated. The relative reflectance difference across the samples was calculated for cross-polarized terahertz images using the background reflectance value Rᵣₑₗ obtained from saline soaked gauze using the formula:

$${R}_{rel} = [ ( {R}_{C} / {R}_{B} ) - ( {R}_{N} / {R}_{B} ) ] / {R}_{N} = \frac{{R}_{C} - {R}_{N}}{{R}_{N} \times {R}_{B}} , \qquad ( Eqn . 1 )$$

Where R_{C} and R_{N} are the reflectance values of cancer and normal colon samples, respectively and R_{B} represents the reflectance from background (saline filled gauze). The normalized relative reflectance difference acquired in this study from fresh colonic tissues using the above equation was 7.73%, which is consistent with the values obtained in previous ex vivo free space terahertz imaging studies.

Some embodiments use a continuous-wave (CW) THz radiation source, as opposed to pulsed THz sources, which are conventionally used in THz imaging. A CW THz radiation source emits THz radiation with a relatively narrow bandwidth, e.g., on the order of MHz. THz imaging systems that use CW sources operate in the frequency domain, as opposed to time domain pulsed source systems. CW frequency domain systems offer the advantage of being lower cost, having simpler data analysis, and faster data acquisition rates. In addition, CW systems provide spectrally selective high-resolution imagery with high signal-to-noise values relative to pulsed systems.

There are many possible applications for THz endoscopic devices. One area of application is colorectal cancer screening. Conventional colonoscopy, the current standard of care for colorectal cancer, relies entirely on the visual inspection by a physician. During colonoscopy, if a polyp or tumor is found, a biopsy or polypectomy may be performed to determine the abnormality presence. Since 85% of all cancers are difficult to detect in early stage, clinicians recur to biopsy excisions for histopathology examination, which is a time consuming process. Since, terahertz radiation is nonionizing and offers intrinsic contrast between normal and abnormal tissues, with embodiments of the present application, quantitative screening for colorectal cancer is possible.

While colorectal cancer screening is one application of embodiments of the present application, it is not the sole application. Any screening or diagnostic that requires terahertz reflectivity data from organs and/or tissue only accessible via endoscope would benefit from THz endoscopic systems according to embodiments of the present application.

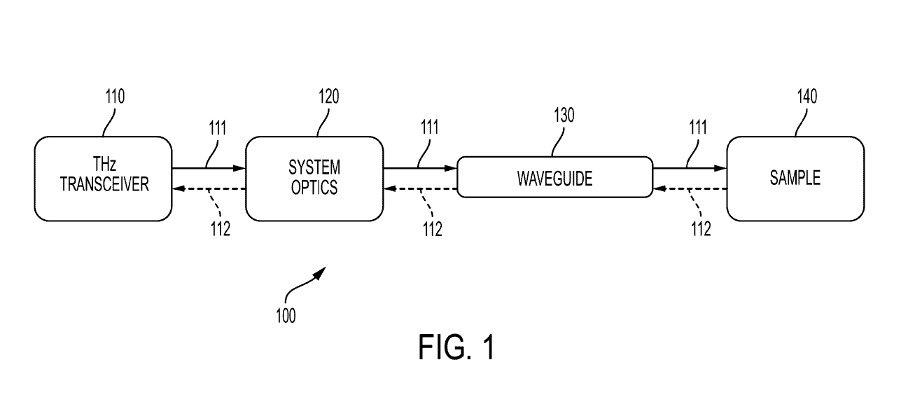

FIG. 1 is a schematic diagram of a THz endoscopic system 100 according to some embodiments. The THz endoscopic system 100 includes three main sections: a THz transceiver 110, system optics 120 and waveguide 130. However, some embodiments may not include all three sections. For example, some embodiments may not include the THz transceiver 110, which may be provided separately, and may only include the system optics 120 and waveguide 130. FIG. 1 also illustrates a sample 140, which is typically provided by the user of the endoscopic system 100. Any suitable sample may be used. In some embodiments, the sample may be the tissue of a human or animal. For example, the sample may be internal tissue of a human that is not readily observed without an endoscope.

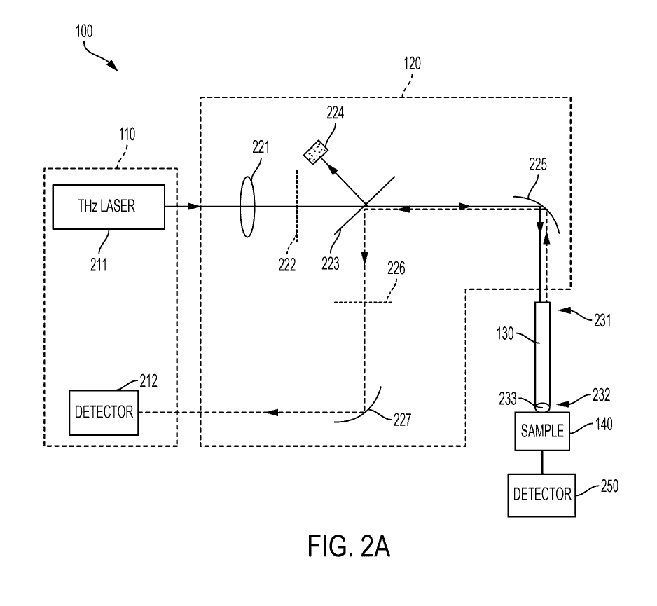

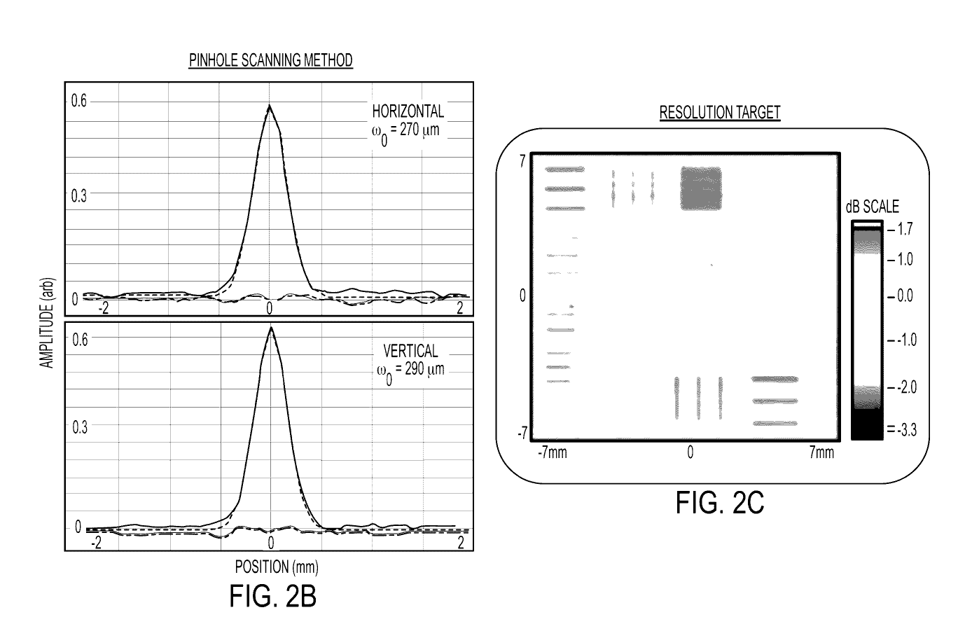

The THz transceiver 110 is a system that both generates THz radiation 111 and detects reflected THz radiation 112 reflected from the sample. While FIG. 1 illustrates the THz transceiver 110 as a single system, embodiments are not so limited. For example, as described below in connection with FIG. 2A, the THz transceiver 110 may be two or more separate components that are located remotely from one another. In some embodiments, the THz transceiver 110 includes a THz radiation source for generating THz radiation 111, and a THz radiation detector for detecting THz radiation 112 reflected from the sample 140.

In some embodiments, the THz transceiver 110 generates a continuous-wave THz radiation output. By way of example, and not limitation, the frequency of the generated THz radiation may fall within a frequency range from 0.2-1.5 THz with a frequency bandwidth of 40-60 GHz. The THz transceiver 110 may be rack-mountable, operate at room-temperature and be low maintenance such that it is possible to use in a clinical setting. While the THz transceiver 110 may be capable of generating a relatively large bandwidth, at any given time, the THz transceiver 110 emits THz radiation with a bandwidth on the order of a MHz. The high bandwidth allows for range gating and other noise reduction techniques, as described below.

The system optics 120 are located between the THz transceiver 110 and the waveguide 130 and serve at least three functions. First, the system optics direct the THz radiation 111 from the THz transceiver 110 to the waveguide 130. Second, the system optics 120 direct the reflected THz radiation 112 from the sample into the waveguide 130, and from the waveguide 130 to the THz transceiver 110. Third, the system optics 120 include optics for isolating the reflected THz radiation 112 from other sources of radiation that add noise to the detected signal.

In some embodiments, the system optics 120 include one or more THz lenses for focusing the terahertz radiation emitted from the THz transceiver 110 to ensure an efficient coupling of terahertz radiation into the waveguide. Focusing the terahertz radiation may, for example, shape the mode profile of the beam such that coupling to the waveguide is optimized. The system optics 120 may also include one or more mirrors for directing the terahertz radiation to and from the waveguide 130. In some embodiments, the mirrors may be curved to shape the mode profile of the THz radiation further. For example, off-axis parabolic mirrors may be used. In some embodiments, one or more polarizers may be used to isolate the reflected THz radiation 112 from other radiation present in the system.

In some embodiments, the waveguide 130 is a flexible, hollow, metal-coated polymer tube with low propagation and bending losses along with Gaussian mode preservation. The waveguide 130 receives THz radiation 111 from the system optics and guides the radiation to the sample. In some embodiments, the endoscopic system includes a lens attached to the waveguide output end to obtain a focused beam of THz radiation. The lens may be, for example, a hyper hemispherical lens (HHS), which is the shape of an extended hemi sphere. In some embodiments, the transmitted THz radiation from the waveguide may be focused to a substantially diffraction-limited spot size that is free of spherical aberration and coma.

Finally, the THz endoscopic system 100 of FIG. 1, with no optics aligned after the distal transmission end of the waveguide, works in reflection modality by transmitting the THz radiation through the waveguide and collecting the reflected THz radiation 112 from the sample 140. The reflected THz radiation is guided by waveguide 130 from the sample 140 to the system optics 120. In some embodiments, the transmission losses due to the fluids surrounding the distal end of the endoscope are mitigated by including a nanostructured hydrophobic surface on the lens. In some embodiments, to reduce the back reflection from the lens surface an anti-reflective coating can be included on one or more surfaces of the lens surface.

FIG. 2A is a more detailed schematic of a THz endoscopic system 100 according to some embodiments. The THz transceiver 110 and the system optics 120 are indicated by the dashed lines encompassing the components of each respective device. THz transceiver 110 comprises a THz radiation source 211 and a THz radiation detector 212. In some embodiments, the THz radiation source 211 generates CW THz radiation. Any suitable CW THz radiation source may be used. For example, the THz radiation source 211 may be a CO₂ pumped far-infrared gas lasers. The THz radiation detector 212 may be any suitable detector that converts received THz radiation into an electrical signal that may be analyzed by, e.g., an electronic circuit or a computer system. For example, the THz radiation detector 212 may be one or more liquid Helium cooled silicon bolometers.

While the aforementioned choice of THz radiation source 211 and THz radiation detector 212 offers flexibility in system design and implementation in a laboratory setting, a different combination of source/detectors may be better suited in a clinical environment. For the clinical environment, the THz radiation source 211 and THz radiation detectors 212 is preferably compact, operates at room-temperature and is low-maintenance. For example, the THz radiation source 211 may be a solid-state frequency multiplier chain which is more suitable for clinical development. These systems may have a frequency bandwidth of ˜40 GHz and may operate at center frequency ranging from 0.1 to 1.5 THz. The THz radiation detectors 212 may include heterodyned diodes and may offer a dynamic range >120 dB. For example the dynamic range may be between 120 dB and 150 dB. The THz radiation detectors 212 may be fully polarimetric and measure both signal amplitude and phase (unlike bolometers which only measure signal intensity). In addition, heterodyned diodes may be faster than bolometers. For example, in some embodiments, images may be taken at a frame rate of 2 frames/second utilizing heterodyned Schottky diodes at terahertz frequencies. One of skill in the art would recognize that embodiments are not limited to any particular THz radiation source 211 or THz radiation detector 212.

While the aforementioned choice of THz radiation source 211 and THz radiation detector 212 offers flexibility in system design and implementation in a laboratory setting, a different combination of source/detectors may be better suited in a clinical environment. For the clinical environment, the THz radiation source 211 and THz radiation detectors 212 is preferably compact, operates at room-temperature and is low-maintenance. For example, the THz radiation source 211 may be a solid-state frequency multiplier chain which is more suitable for clinical development. These systems have a frequency bandwidth of ˜40 GHz and may operate at center frequency ranging from 0.1 to 1.5 THz. The THz radiation detectors 212 may include heterodyned diodes and may offer a dynamic range >120 dB. For example the dynamic range may be between 120 dB and 150 dB. The THz radiation detectors 212 may be fully polarimetric and measure both signal amplitude and phase (unlike bolometers which only measure signal intensity). In addition, heterodyned diodes may be faster than bolometers. For example, in some embodiments, images may be taken at a frame rate of 2 frames/second utilizing heterodyned Schottky diodes at terahertz frequencies. Embodiments are not limited to any particular THz radiation source 211 or THz radiation detector 212.

The system optics 120 include a variety of components. A THz lens 221 receives the THz radiation from the THz radiation source 211 and directs the THz radiation to a first polarizer 222. The THz lens 221 acts on the THz radiation to shape the beam expansion profile of the beam received from the THz radiation source 211. In some embodiments, the beam exiting from the THz radiation source 211, which may be a CW THz laser, has a beam waist that is a few millimeters in diameter and expands fairly rapidly in free space. Accordingly, the THz lens 221 collimates the beam. Any suitable THz lens 221 may be used. For example, the THz lens 221 may be a plano-convex or biconvex lens. The THz lens 221 may also be made from any suitable material. For example, the THz lens 221 may be made from a polymer such as polymethylpentene (TPX) or High-density polyethylene (HDPE).

The first polarizer 222 is oriented in a first direction such that it transmits THz radiation, received from the THz lens 221, which is linearly polarized in a first direction. The polarizer cleans up the polarization of the transmitted THz radiation such that it is linearly polarized to a high degree. In some embodiments, the THz radiation source 211 may be sufficiently polarized such that polarizer 222 is not necessary and may be omitted. Any suitable polarizer that operates in the THz regime may be used. For example, a wire grid polarizer may be used. If the THz radiation source 211 is, by way of example and not limitation, vertically polarized then the polarizer 222 is oriented such that only vertically polarized radiation passes through and horizontally polarized radiation is blocked. In other embodiments, polarizations other than linear polarizations may be used. For example, circularly polarized light may be used. In such embodiments, the first polarizer 222 may include a birefringent material, such as quartz, to rotate the polarization of the THz radiation after it encounters a linear polarizer.

The polarized THz radiation that passes through polarizer 222 then encounters beam splitter 223, which splits the input THz radiation into two components. In some embodiments, 50% of the received THz radiation is transmitted and 50% of the received THz radiation is reflected. The reflected component of the THz radiation is disposed of by being absorbed by THz absorber 224. The transmitted component of the THz radiation is coupled into the waveguide 130 for use in imaging.

The THz radiation that is transmitted through the beam splitter 223 then encounters a curved mirror 225 for shaping the mode profile of the beam to decrease the amount of loss when coupling the radiation into waveguide 130. Any suitable curved mirror 225 may be used. In some embodiments, a front-surface silver coated off-axis parabolic mirror 225 is used to focus the collimated THz radiation beam to a small spot size, e.g., of the order of THz wavelength, and to correct for spherical aberration. The parabolic mirror 225 transforms an incoming plane wave of THz radiation traveling along the axis into a spherical wave converging toward the focus of the parabolic mirror. In some embodiments, instead of a curved mirror a series of flat mirrors and lenses may be used to shape the mode profile of the THz radiation.

The THz radiation is then coupled into a waveguide 130. Any flexible waveguide suitable for guiding THz radiation may be used. In some embodiments it is preferable to use a flexible, hollow, metal-coated waveguide with low propagation and bending losses along with Gaussian mode preservation. To obtain flexibility, a polycarbonate tube may be used as the base tubing for waveguide 130. For endoscopic applications, the guided THz radiation should be confined within the waveguide. To confine the terahertz radiation inside the tube, a highly reflective metal (such as silver or gold) is coated on the inner surface of the polycarbonate tube using, e.g., a liquid phase chemical deposition process. In some embodiments, to obtain increased transmission (and, therefore, low-loss) through the waveguide, and to attain maximum coupling efficiency, the ratio of beam size and waveguide diameter is between 0.6 and 0.85, for example 0.77. This ratio can be achieved by adjusting the parameters of at least mirror 225. One of skill in the art would recognize that these values are for illustrative purposes and other embodiments, such as embodiments utilizing circular polarization may use other values.

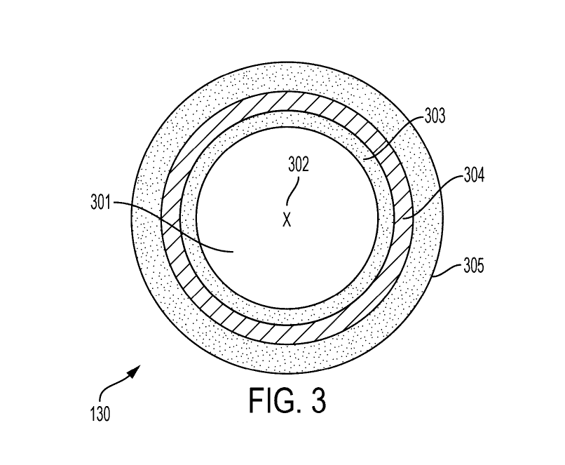

In some embodiments, the waveguide 130 is a low loss waveguide with less than 2 dB/m of loss and preferably less than 1 dB/m of loss. The total transmission loss of a flexible waveguide increases as a function of bending angle and bend radius. For example, the transmission loss of a 2.5° bent waveguide, to scan 2 cm×2 cm area, increases from 1.62 to 1.72 dB/m. The waveguide 130 may be any length, but should be long enough to reach the desired sample. For example, the waveguide 130 could be between 40 cm and 100 cm long, or approximately 45 cm, plus or minus 10%. A cross section of a waveguide 130 according to some embodiments is illustrated in FIG. 3. The waveguide 130 has a hollow core 301 that is centered upon a central axis 302. The waveguide 130 comprises a plurality of layers, each layer located at a corresponding radial distance from the central axis. In some embodiments, an optional nonmetallic layer 303 is the inner most layer located at a first radial distance from the central axis 303. The nonmetallic layer 303 may be formed from any suitable material. For example, the nonmetallic layer 303 may be a polymer with a low extinction coefficient, such as polystyrene or polyethylene/polytetrafluoroethylene or Teflon. A metallic layer 304 is located adjacent to the nonmetallic layer 303 at a second radial distance that is larger than the first radial distance. The metallic layer 304 may be formed from any suitable material that is highly reflective at THz wavelengths. For example, the metallic layer 304 may be formed from gold, aluminum, copper or silver. The metallic layer should have a thickness that is small enough to maintain flexibility of the endoscope and large enough to maintain the field confinement. For example, the thickness may be approximately 1-2 micrometers. A nonmetallic layer 305 is located adjacent to the metallic layer 304 at a third radial distance that is larger than the second radial distance. The nonmetallic layer 305 may be formed from any suitable flexible material. For example, the nonmetallic layer 305 may be formed from a polymer such as polycarbonate.

The above mentioned radial distances should be small enough for endoscopic applications. For example, the hollow core 301 may have a diameter of approximately 1-2 mm.

The waveguide 130 has two ends: a proximal end 231, where THz radiation from the system optics 120 is coupled into the waveguide 130, and a distal end 232, where THz radiation is emitted for the purpose of irradiating the sample 140. The distal end 232 may include a lens 233 for focusing the light on the sample 140. Any suitable lens may be used, such as a hyper hemispherical (HHS) lens, a hemispherical lens or a ball lens. In some embodiments, the lens 233 may be a hyper hemispherical (HHS) lens that is attached to the distal end of the waveguide. In some embodiments, the HHS lens may provide a large angle of view. The HHS lens results in a THz radiation beam with a small spot size that is substantially free from diffraction effects. Due to the extended hemispherical shape of the HHS lens 233, the divergence of the THz beam decreases and eventually leads to a collimated beam. The HHS lens 233 may be free of circular coma and spherical aberration such that it produces a fundamental Gaussian THz beam with a small waist (e.g., with a size of λ/2) located behind the lens (˜1 to 2λ distance).

In some embodiments, the lens 233 includes a hydrophobic surface to reduce transmission loss and create a clear path through fluids that may surround the distal end of the endoscope when in contact with the sample 140. Any suitable hydrophobic surface may be used. For example, a nanostructured hydrophobic surface formed using a soft nanolithography technique may be used.

In some embodiments, the lens 233 may also include at least one anti-reflection (AR) coating. The THz radiation transmits through the hollow waveguide and then at the waveguide output (distal) end, without an AR coating, the THz radiation may be partially reflected due to the HHS lens surface. To minimize the back reflection from the lens an anti-reflective coating may be deposited on the first (curved) surface of the lens. However, to minimize the astigmatism resulted from the HHS lens and to overcome the reflection while collecting the back reflected terahertz signal from the sample an anti-reflection coating may also be deposited on the second (flat) surface of the lens.

THz radiation emitted from the distal end 232 of the waveguide 130 interacts with the sample 140 and a portion of the radiation, referred to as the reflected THz radiation, reflects off the sample 140 and is coupled back into the waveguide 130 via lens 233. The reflected THz radiation travels the opposite direction through the waveguide 130 as the THz radiation used to irradiate the sample 140 is guided using the same single channel as is used to direct the THz radiation to the sample 140 in the first place. When the reflected THz radiation reaches the proximal end 231 of the wave guide 130, it is coupled into free space where it is collimated by the curved mirror 225. For example, the reflected THz radiation may couple to free space as a substantially spherical wave from the proximal end 231 located at the focal point of the off-axis parabolic mirror 225, which results in the reflected THz radiation forming a collimated beam propagating back towards beam splitter 223.

At beam splitter 223, a first portion of the reflected THz radiation from the sample 140 is reflected and a second portion of the reflected THz radiation is transmitted through the beam splitter 223. The reflected portion is directed towards a second polarizer 226. Any suitable polarizer that operates in the THz regime may be used. For example, a wire grid polarizer may be used. The second polarizer 226 may be oriented in any suitable direction. In some embodiments, co-polarized reflected THz radiation is measured by orienting the second polarizer 226 in the same direction as the first polarizer 222. In this way, only reflected THz radiation with the same polarization as the incident THz radiation 111 is used to irradiate the sample 140 is detected by detector 212 and analyzed. All other radiation is not transmitted through the second polarizer 226. In other embodiments, cross-polarizer reflected THz radiation is measured by orienting the second polarizer 226 in a direction orthogonal to the orientation of the first polarizer 222. In this way, only reflected THz radiation that is orthogonally polarized relative to the polarization of the THz radiation 111 is used to irradiate the sample 140 is detected by detector 212 and analyzed. All other radiation is not substantially transmitted through the second polarizer 226 and is instead reflected and/or absorbed by the second polarizer 226. Embodiments are not limited to using crossed polarizers. In some embodiments, the polarizers may be oriented in a non-parallel arrangement such that the polarization that transmits through the first polarizer 222 is different from the polarization transmitted through the second polarizer 226.

After the second polarizer 226 is a curved mirror 227 configured to focus the reflected THz radiation on the active area of detector 212. Similar to curved mirror 225, in some embodiments, curved mirror 227 may be an off-axis parabolic mirror. Alternatively, one or more flat mirrors and lenses may be used to direct and focus the reflected THz radiation onto the detector 212.

The reflected signal detected by detector 212 may be isolated from other signals, such as signals associated with background radiation, in any suitable way. In some embodiments, the isolation may be done optically by isolating the reflected THz radiation from other radiation. Alternatively, or additionally, the isolation may be done electronically by analyzing the signal from detector 212 in a particular way.

In some embodiments, the reflected THz signal is isolated from other signals using polarizer 222 and polarizer 226 in a crossed (or different) configuration.

In some embodiments, the reflected THz signal is isolated from other signals using an active THz radiation source 211 that is frequency chirped and a coherent detection scheme that allows for range gating the acquired signal. These techniques reduce unwanted signals generated from sources other than THz radiation reflected by the sample 140 and also reduce system noise in the generated images. By way of contrast, a purely single frequency system cannot differentiate reflections from different surfaces. Accordingly, the entire radiation backscattered to the detector through system optics is detected, including back-reflections from system optics, and considered as part of the signal. However, in embodiments using frequency chirping or bandwidth (BW) to the THz radiation source, different scattering surfaces can be isolated. After collecting data with detector 212 in the frequency domain (as the ‘chirp’ sweeps through frequencies), the Fourier transform of the acquired data may be used to obtain range resolution. The highest range resolution (ΔR) possible is a function of the total sweep bandwidth (BW) of the THz radiation source 211:

ΔR=c/2BW (Eqn. 2).

Thus, larger bandwidth allows for separation of surfaces that are closer together. Because the distance from the detector 212 to the sample 140 is known, the data obtained by detector 212 can be filtered, in the Fourier domain, to include only the data associated with reflections that are related with the sample 140. After filtering in the Fourier domain, the resulting data may be inverse Fourier transformed so that the isolated data may be analyzed.

Any suitable frequency-chirped source 211 may be used to generate frequency-chirped THz radiation. In some embodiments, the frequency-chirping is generated by sweeping a synthesizer frequency prior to up conversion in solid-state source. Alternatively, the frequency-chirping may be created by mixing a laser source with a microwave source in a mixer (e.g., a Schottky Diode).

In some embodiments, the reflected THz signal is isolated from other signals by filtering the detected signal from the detector 212 at particular frequencies that match the frequency of the THz radiation source 211. For example, the emission frequency of the frequency-chirped THz radiation source 211 is known at any given time. Thus, the detected signal at a corresponding time should be phase and amplitude matched with the emitted THz radiation from the THz radiation source 211.

In some embodiments, the reflected THz signal may be isolated from other signals by calibrating out the detected signals associated with stray radiation. For example, if a frequency-chirped THz radiation source 211 is used, the detected signal associated with the reflected THz radiation should change along with the emitted signal. Accordingly, any signal that does not change over time as the source frequency is changed may be considered background signal from some other source. This static signal, or an average of the static signal, may be subtracted from the detected signal to remove the influence of this stray radiation from the data analysis.

FIG. 2B illustrates the results of a beam waist measurement of the THz radiation of the system 100 illustrated in FIG. 2A. The measurements were obtained in both transmission and reflection modalities using a pinhole scanning method and resolution target. The measurement used a 250 μm hole in an aluminum plate as the aperture and displays the transmitted terahertz power as a function of position. The resolution of the profile is determined by the size of the aperture and the scan stage step resolution. By fitting the Gaussian spatial profile to the transmitted curve, horizontal and vertical beam waists of 270 μm and 290 μm were obtained. To test the resolution of the terahertz endoscopic system in reflection modality, a standard positive 1951 USAF resolution target (made by plating chrome on a soda lime glass substrate) was used (see FIG. 2C). Since the 4th and 5th elements of group 1 are resolved in FIG. 2C, the horizontal and vertical resolutions were calculated as 353 μm and 315 μm, respectively.

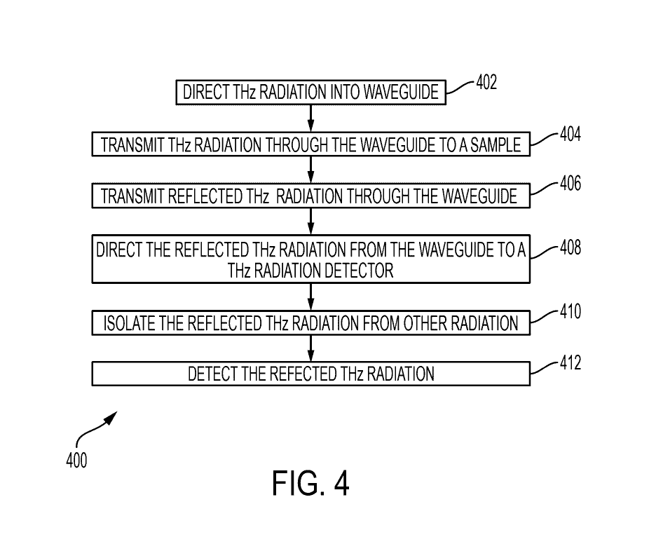

FIG. 4 illustrates a flow chart of method 400 for performing THz endoscopy according to some embodiments. At act 402, THz radiation is directed into a waveguide. This may be done in any suitable way. For example, any of the techniques using system optics 120, including off-axis parabolic mirrors, polarizers and beam splitters may be used to direct the THz radiation. Additionally, the THz radiation may originate from any suitable THz radiation source, such as the frequency-chirped sources described above.

At act 404, the THz radiation is transmitted from a proximal end of the waveguide to a distal end of the waveguide. The THz radiation is coupled out of the waveguide via, as described above, a lens attached to the distal end of the waveguide such that the THz radiation is focused onto a sample.

At act 406, reflected THz radiation from the sample is coupled into the waveguide via the same lens that coupled the THz radiation from the waveguide to the sample. The reflected THz radiation is transmitted from the distal end of the waveguide to the proximal end of the waveguide.

Any suitable waveguide may be used. For example, the waveguides described above in connection with FIG. 2A and FIG. 3 may be used in some embodiments. Preferably, the endoscopic system uses a single channel waveguide that transmits the THz radiation to the sample and collect the reflected THz radiation from the sample using the same channel.

At act 408, the reflected THz radiation is directed from the proximal end of the waveguide to a THz radiation detector. This may be done in any suitable way. For example, the reflected THz radiation may be directed using the system optics 120 described above.

At act 410, the reflected THz radiation is isolated from other radiation. This may be done in any suitable way. For example, as described above, crossed polarizers may be used to isolate the signal. Additionally, the THz radiation may be isolated from other radiation after detection by analyzing the data acquired by the detector in any suitable way, for example, as described above.

At act 412, the reflected THz radiation is detected using a THz radiation detector. Any suitable THz radiation detector may be used. For example, the heterodyned diodes or bolometers described above may be used.

### Example Experimental Use and Results

The reflectance of normal and cancerous colon tissue was investigated using a CO₂ optically pumped infrared gas laser operating at 584 GHz with an output power of 33 mW. The experimental setup was similar to the apparatus illustrated in FIG. 2. The beam exiting the terahertz radiation source was collimated using a 61 cm focal length TPX lens and then passed through a wire grid polarizer, a 50-50 Mylar beam splitter, and finally focused to 0.68 mm spot size using a 9 cm focal length off-axis parabolic mirror. A low loss, flexible terahertz waveguide is used for beam confinement. To obtain flexibility, a polymer (polycarbonate) with low inner surface roughness was used as the base tubing. To confine the terahertz radiation inside the tube, a 99% reflective metal (e.g., silver or gold) was coded on the inner surface of the polycarbonate tube using a liquid phase chemical deposition process. To obtain low loss transmission through the waveguide, and to attain maximum coupling efficiency, the ratio of beam size and waveguide diameter was maintained at 0.77 by adjusting the off axis parabolic mirror parameters. The metal coated waveguide is capable of simultaneous dual-frequency confinement at 1.4 THz and 584 GHz, confirming the ability for these waveguides operate at multiple frequency ranges.

The endoscopic system included a flexible 4 mm diameter metal coated waveguide to transport the terahertz radiation and 5 mm diameter Z-cut courts hyper-hemispherical lens attached at the output of the waveguide to obtain a diffraction limited beam waste of 0.28 mm, located behind the lens which is free of circular coma and spherical aberration.

An automated X-Y scanning stage was used to raster scan the sample in the imaging plane with the scanning resolution of 0.1 mm, while the transmitted part of the beam was focused into a silicon bolometer detector 250 (illustrated in FIG. 2A) using a 7 cm focal length high density polyethylene lens. The dwell time per location in the image was approximately 150 ms. The terahertz radiation was optically modulated with 80 Hz frequency, which served as a reference for a lock-in amplifier. The reflected terahertz radiation reflected from the sample was focused into a terahertz radiation detector located in the reflection arm using a 9 cm focal length off-axis parabolic mirror. Co-polarized and cross-polarize terahertz images were obtained by placing appropriately oriented polarizer in the reflection arm.

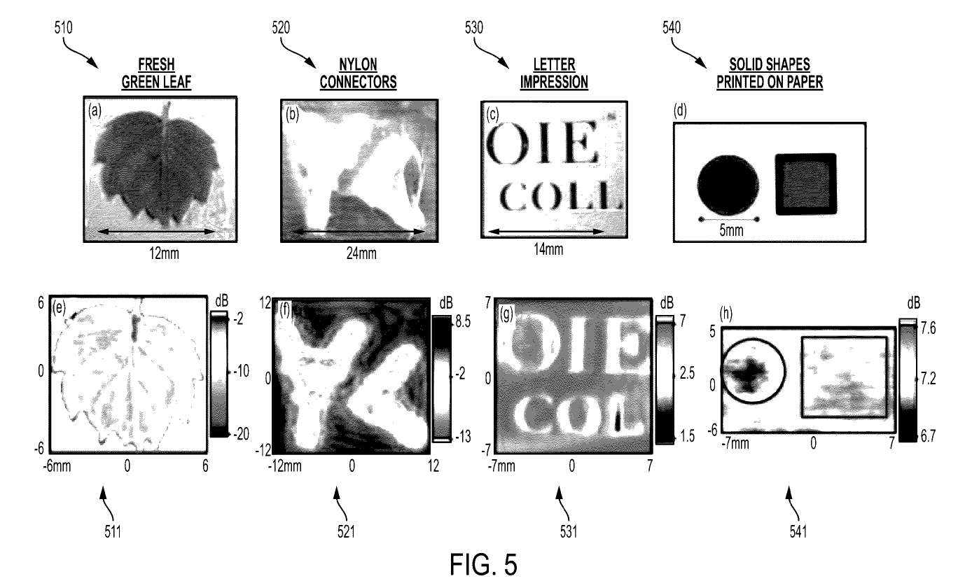

The THz transmission imagery of various objects is shown in FIG. 5. Image 511 shows the terahertz transmission image of a small leaf (half the size of a penny), mounted on a 1 mm thick glass slide. The generated terahertz contrast of the leaf was primarily from the water content within the veins. The structure in the transmission image correlated well with the digital photograph shown in image 510. Therefore, the THz transmission image of the leaf is a good indicator of the endoscopic system's high resolution. Image 521 shows the THz transmission image of nylon wye and elbow connectors mounted between two paraffin sheets. The stress in the paraffin film from stretching can be seen clearly in the THz transmission image. A digital photograph of the nylon is shown in image 520. 531 and 541 depict the THz transmission images of letter impressions on a polymer sheet and colored solid shapes printed on a normal printing paper (a digital photograph of the letter impression is shown in image 530 and a digital photograph of the solid shapes printed on paper are shown in image 540). These images substantiates the potential use of terahertz radiation, which can penetrate through materials such as a paper envelope, to reveal the contents.

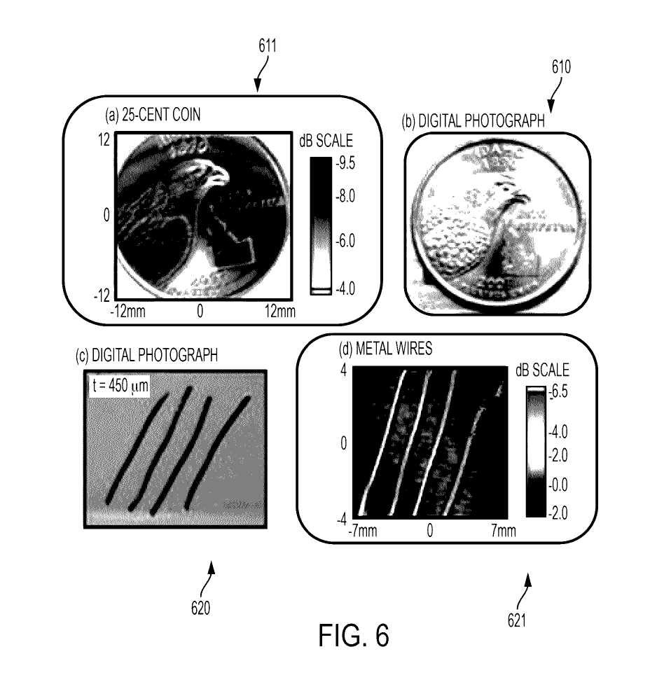

The THz reflection imager of various objects is shown in FIG. 6. The reflection imagery of four copper wires (image 621) mounted adjacent to each other and a 25-cent coin (image 611) were acquired. A digital photograph of the wires used is shown in image 620. Both the terahertz images were obtained by collecting the entire signal remitted from the sample at 584 GHz frequency. The THz reflection imagery of the coin shows the structural information of its surface, which correlates well with the digital photograph shown in inset image 610. On the other hand, both the width and edges of 450 μm thick wires were clearly visible in inset image 621. Therefore, the endoscopic system's imaging resolution in reflection modality is apparent from FIG. 6.

Imaging of cancerous tissue was also performed using the apparatus of FIG. 2A. Fresh thick excess colon specimens obtained from University of Massachusetts Memorial Hospital under an Institutional Review Board approved protocol were used as samples. The thickness of the specimens varied between 4 to 6 mm, with lateral dimensions being 10 to 15 mm. For terahertz imaging, the tissue specimens were mounted in an aluminum sample holder, with a 7.5 cm×2.5 cm opening, covered with a 1.55 mm thick z-cut quartz slide. To prevent tissue dehydration during the imaging procedure, the specimens were covered with wet gauze soaked in pH-7.4 balanced saline. The tissue specimens were imaged fresh and then fixed with 10% neutral-buffered formalin solution and stored in the refrigerator at 4° C. for 96 hours. In total we measured six specimens (three all-cancerous and three all-normal) from three subjects. The terahertz images were then calibrated against the full-scale return from a flat front-surface gold-coated mirror to determine the reflectance. The images were plotted in logarithmic space and the off sample areas were removed in the post processing.

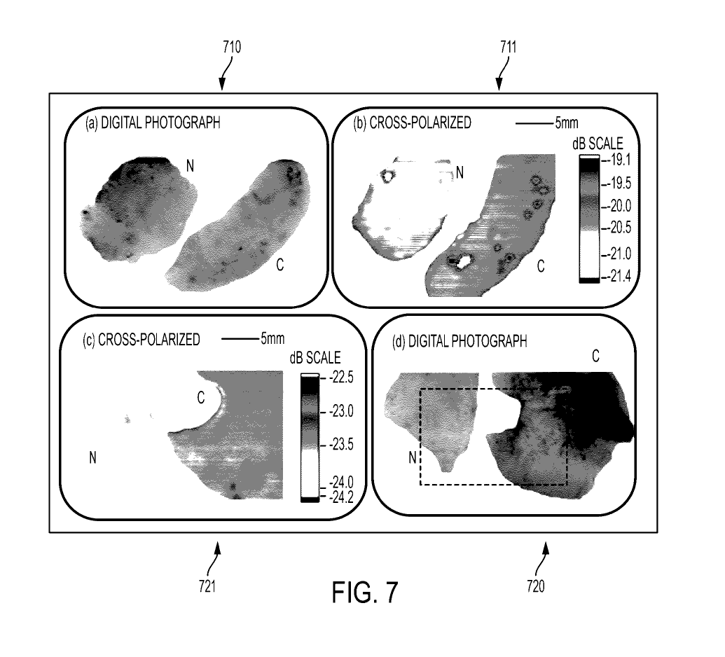

FIG. 7 shows digital photographs and cross-polarized terahertz reflectance images of two colonic tissue sets. Image 710 is a digital photograph of a cancerous and a normal human colonic formalin fixed tissue and image 711 is the THz image of the same samples. Image 720 is a digital photograph of a cancerous and a normal fresh human colonic tissue and image 721 is the THz image of the same samples. An intrinsic contrast was observed between cancerous (labeled C) and normal (labeled N) colonic tissues with cancerous specimens showing higher reflectivity values. However, as shown in image 710, the normal specimen (labeled N) shows higher reflectance values in certain regions within the tissue (shown in a darker shade) that is comparable with the adjacent cancerous tissue. Also, in contrast to the histology, the cancerous colonic tissue exhibited lower reflectance values (the lighter shade region), indicating the presence of normal tissue region. The cross-polarized reflectance from fresh normal samples was found to be between 0.38 to 0.41%, whereas for cancerous specimens it was between 0.44 to 0.46%. In case of formalin fixed samples, the cross-polarized reflectance of normal tissue varied from 0.85 to 0.88%, while for cancer specimens it varied from 0.92 to 0.96%. Furthermore, the cross-polarized reflectance of normal specimen varied from −22 to −24.5 dB and the cancerous specimen from −20.6 to −21.3 dB, exhibiting the overlap of reflectivity values. This may have resulted from the insufficient signal-to-noise ratio (SNR), 26 dB, of the imaging system.

Analysis of the reflectivity data obtained from formalin fixed and fresh samples of FIG. 7 show that cancerous tissue had higher reflectivity than normal tissue. An increased reflection from the cancerous region may possibly be attributed to the greater scattering (refractive index fluctuation) resulting from increased vasculature, lymphatic systems, and other structural changes in diseased tissues. The normalized relative reflectance difference acquired in this study from colonic tissues at 584 GHz was found to be 7.73%. During formalin fixation, the water content (with a refractive index of 2.4 at 584 GHz) in the tissue will be replaced by formalin solution that has a lower refractive index resulting in lower reflectance values. The relative reflectance difference obtained in this study from fixed tissues is 5.31%. Therefore, this work using polarization sensitive detection technique furthers the potential application of cross-polarized terahertz imaging to biomedical areas that require endoscopic access.

### CONCLUSION

Embodiments of the present application have been used to demonstrate a continuous-wave terahertz endoscopic system for cancer detection. Some embodiments use a single-channel to transmit and collect the back reflected intrinsic terahertz signal from the sample and are capable of operation in both transmission and reflection modalities. By implementing cross-polarized reflectance THz imaging, a contrast between normal and cancerous colonic tissue was shown by rejecting specular reflections. The analysis indicates that the imaging system and polarization techniques are capable of registering reflectance differences between normal and cancerous colon. Thus, in some embodiments, THz reflectivity data from previously inaccessible organs may be used to significantly increase the overall impact of terahertz imaging for biomedical detection/screening applications

Having thus described several aspects of several embodiments, it is to be appreciated that various alterations, modifications, and improvements will readily occur to those skilled in the art. For example, the inventors have also recognized and appreciated that both the portion of the reflected THz radiation reflected from polarizer 226 in FIG. 2 and the portion of the reflected THz radiation transmitted through polarizer 226 may both be detected by separate detectors. In this way, both co-polarized and cross-polarized signals may be detected simultaneously.

Such alterations, modifications, and improvements are intended to be part of this disclosure, and are intended to be within the spirit and scope of the invention. Further, though advantages of some embodiments are indicated, it should be appreciated that not every embodiment will include every described advantage. Some embodiments may not implement any features described as advantageous herein and in some instances. Accordingly, the foregoing description and drawings are by way of example only.

Also, some methods or processes outlined herein may be coded as software that is executable on one or more processors that employ any one of a variety of operating systems or platforms. For example, the Fourier transform techniques and signal analysis described above may be performed by discrete circuits, FPGAs, ASICS, or software running on a computer system. Additionally, such software may be written using any of a number of suitable programming languages and/or programming or scripting tools, and also may be compiled as executable machine language code or intermediate code that is executed on a framework or virtual machine.

In this respect, the invention may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the invention discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the present invention as discussed above. As used herein, the term “computer-readable storage medium” encompasses only a computer-readable medium that can be considered to be a manufacture (i.e., article of manufacture) or a machine. Alternatively or additionally, the invention may be embodied as a computer readable medium other than a computer-readable storage medium, such as a propagating signal.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Various aspects of the above embodiments may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which several examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. A terahertz endoscopic system, comprising:
a waveguide configured to:
transmit terahertz radiation from a first end of the waveguide to a second end of the waveguide proximate to a sample; and
transmit reflected terahertz radiation from the second end of the waveguide to the first end of the waveguide, wherein the reflected terahertz radiation is a portion of the terahertz radiation reflected by the sample towards the second end of the waveguide; and
system optics configured to:
direct the terahertz radiation from a radiation source into the first end of the waveguide;
isolate the reflected terahertz radiation from other radiation; and
direct the reflected terahertz radiation from the first end of the waveguide to a terahertz radiation detector.

2. The terahertz endoscopic system of claim 1, wherein the system optics comprise:
a first polarizer, positioned between the radiation source and the waveguide, configured to transmit radiation a first polarization; and
a second polarizer, positioned between the waveguide and the terahertz radiation detector, configured to transmit radiation of a second polarization that is different from the first polarization.

3. The terahertz endoscopic system of claim 2, wherein the system optics further comprise:
a beam splitter configured to direct the reflected terahertz radiation from the first end of the waveguide to the second polarizer.

4. The terahertz endoscopic system of claim 1, wherein the radiation source is located remotely from the second end of the waveguide.

5. The terahertz endoscopic system of claim 1, wherein the terahertz radiation detector is located remotely from the second end of the waveguide.

6. The terahertz endoscopic system of claim 1, wherein the waveguide is a flexible hollow-core waveguide comprising a hollow core.

7. The terahertz endoscopic system of claim 6, wherein the waveguide comprises:
a metallic layer positioned a first radial distance from a center of the hollow core such that the metallic layer radially surrounds the hollow core.

8. The terahertz endoscopic system of claim 7, wherein the waveguide comprises:
a first nonmetallic layer positioned a second radial distance from a center of the hollow core such that the first nonmetallic layer radially surrounds the hollow core, wherein the second radial distance is greater than the first radial distance.

9. The terahertz endoscopic system of claim 8, wherein the first nonmetallic layer comprises a polymer.

10. The terahertz endoscopic system of claim 9, wherein the polymer comprises polycarbonate.

11. The terahertz endoscopic system of claim 8, wherein the waveguide comprises:
a second nonmetallic layer positioned a third radial distance from a center of the hollow core such that the second nonmetallic layer radially surrounds the hollow core, wherein the second radial distance is less than the first radial distance.

12. The terahertz endoscopic system of claim 11, wherein the second nonmetallic layer comprises a polymer.

13. The terahertz endoscopic system of claim 12, wherein the polymer comprises polystyrene.

14. The terahertz endoscopic system of claim 1, wherein the waveguide comprises a lens at the second end.

15. The terahertz endoscopic system of claim 14, wherein the lens is a hyper hemi-spherical lens.

16. The terahertz endoscopic system of claim 14, wherein the lens comprises a hydrophobic surface.

17. The terahertz endoscopic system of claim 1, wherein the terahertz radiation produced by the radiation source is frequency chirped.

18. A method of performing terahertz endoscopy, the method comprising acts of:
directing terahertz radiation emitted from a radiation source into the first end of a waveguide using system optics;
transmitting terahertz radiation from the first end of the waveguide to a second end of the waveguide proximate to a sample;
transmitting reflected terahertz radiation from the second end of the waveguide to the first end of the waveguide, wherein the reflected terahertz radiation is a portion of the terahertz radiation reflected by the sample towards the second end of the waveguide;
isolating the reflected terahertz radiation from other radiation; and
directing the reflected terahertz radiation from the first end of the waveguide to a terahertz radiation detector.

19. The method of claim 18, wherein the act of isolating the reflected terahertz radiation from the other radiation comprises:
passing the terahertz radiation through a first polarizer before directing the terahertz radiation into the first end of the waveguide, where the first polarizer transmits radiation of a first polarization; and
passing the reflected terahertz radiation through a second polarizer before directing the reflected terahertz radiation to the terahertz radiation detector, wherein the second polarizer is configured to transmit radiation of a second polarization that is different from to the first polarization.

20. The method of claim 18, wherein:
the terahertz radiation produced by the radiation source is frequency chirped; and
the act of isolating the reflected terahertz radiation from the other radiation comprises range gating the signal generated by the terahertz radiation detector.
